(19) United States
(12) Patent Application Publication
(10) Pub. No.: US 2017/0143375 A1
(43) Pub. Date: May 25, 2017

(54) METHODS AND KITS FOR REPAIRING FRACTURED BONES AND JOINTS BY CHEMICAL OR BIOLOGICAL AGENTS

(71) Applicant: Annu H. Navani, Fremont, CA (US)

(72) Inventor: Annu H. Navani, Fremont, CA (US)

(21) Appl. No.: 14/949,886

(22) Filed: Nov. 24, 2015

(51) Int. Cl. A61B 17/56; A61K 9/00

(52) U.S. Cl. CPC A61B 17/56; A61K 9/0019; A61B 2017/564

(57) ABSTRACT

A method for repairing a fractured bone or a joint of a patient by chemical and/or biological agents, includes the steps of, determining an exact section of the fractured bone or the joint to be repaired, selectively delivering a quantity of an anesthetizing composition into skin surrounding the exact section in order for at least a portion of the anesthetizing composition to be absorbed by one or more tissues in vicinity of one or more nerves responsible for transmitting pain impulses from the exact section to brain of the patient in order to thereby selectively and remotely anesthetize at least a portion of the exact section, supervising the exact section in real-time and delivering one or more chemical and/or biological agents into the exact section, and, applying one or more bandages and/or one or more braces on the exact section for proper alignment and fusion.

### BACKGROUND OF THE INVENTION

Field of the Invention

Embodiments of the present invention generally relate to bone and joint repair, and more particularly, to methods and kits for repairing fractured bones and joints by employing chemical and/or biological agents or combinations there of.

Description of the Related Art

Due to physical trauma and/or disease the joints in the limbs of a human are frequently subject to a loss of function. As a result, these joints become increasingly more painful and problematic for the person over a period of time. Currently, surgeons generally have relatively few options for restoring the form and function of the affected joints. Therefore, while performing certain medical procedures, such as reconstructing a joint that has been damaged due to bone or soft tissue trauma, the surgeon may need to fuse the bones of the joint together in a configuration that approximates the natural geometry of the joint. However, many clinical factors including but not limited to patient age, patient weight and general patient health further complicate the task of restoring the affected joints to a fully functional, pain-free condition.

Conventional medical procedure to achieve the objective of fusion of the damaged or arthritic joints is to attach the bones of the joint to a metallic plate that holds the bones in alignment with one another while they fuse together. This is performed by surgeons utilizing the invasive approach which includes the steps of manipulating the tissues and inserting the metallic hardware. However, this procedure leads to prolonged healing process, and long recovery time. In many cases the patient suffers from post-operative pain and lack of function in the joint. Moreover, the metallic devices placed in and around bony and/or joint surfaces causes trauma or irritation to surrounding structures in the body. Many a time, the patient might be allergic to one or more metals and the metallic plate might pose to be a problem.

Currently, many types of hardware are available both for fracture fixation and for the fixation of bones that are to fused. Metal and absorbable materials are routinely used to fixate bone fractures. However, it is important to the successful outcome of the medical procedure that the device is able to generate the compressive forces helpful in promoting bone healing. A disadvantage of mechanical fasteners, however, is that they can damage the tissue or vessel wall when the hardware is deployed. For example, a broken hip bone utilizes a metallic plate and screw of appropriate metal and shape to reconnect the broken members and restore the hip to normal use. In effect, modern surgical practice not only restores or replaces deteriorating bone sections but includes the possible replacement of substantially any bone member or bone portion of the human skeleton. While traveling from one city to another the person is subjected to scanning by an X-ray machine, the presence of such metal in the human being will cause a signal to function in a manner to indicate the presence of metal in or on the person being scanned to detect such presence. This will subsequently alert the security personnels at the airport and generally it is assumed that the individual is carrying a weapon or any other objectionable device. Consequently, the individual who has undergone the surgery has to explain the security personnel at the airport.

However, in other cases the implantable medical devices don't attach effectively. The metallic materials for e.g., stainless steel, titanium based alloys, fluoropolymers, and ceramics do not provide a good substrate for host tissue attachment and ingrowth during the scarring process. Consequently, the ineffective attachment between the device and the host tissue, results in migration of the device within the vessel or tissue in which they are implanted.

Currently, injectable bone cement for filling bone vacuums with mechanical properties that are comparable with the sponge tissue of vertebral bodies are available. The injectable bone cement is composed of an acrylic polymer and a radio-opaque inorganic agent. However, the available injectable bone cement does not propose the use of biocompatible and biodegradable polymeric materials, based on natural polymers, and has components of acrylic nature, with all the problems related to their use.

Accordingly, there remains a need in the medical domain, to develop methods and kits to achieve the same degree of fusion of bones and/or joints without surgical manipulation and incision of skin. The patient will not suffer from post-operative pain and will recover faster. Further, there remains a need to eliminate the need for open surgery and shorten the recovery time for the patient.

### SUMMARY OF THE INVENTION

Embodiments of the present disclosure provide a method for repairing a fractured bone or a joint of a patient by chemical and/or biological agents, including the steps of, determining an exact section of the fractured bone or the joint to be repaired, selectively delivering a quantity of an anesthetizing composition into skin surrounding the exact section in order for at least a portion of the anesthetizing composition to be absorbed by one or more tissues in vicinity of one or more nerves responsible for transmitting pain impulses from the exact section to brain of the patient in order to thereby selectively and remotely anesthetize at least a portion of the exact section, supervising the exact section in real-time and delivering one or more chemical or biological agents or a combination there of into the exact section, and, applying one or more bandages and/or one or more braces on the exact section for proper alignment and fusion.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a flow diagram of a method for repairing a fractured bone or a joint of a patient by chemical and/or biological agents, in accordance with one embodiment of the present invention.

While the present systems and methods have been described herein by way of example for several embodiments and illustrative drawings, those skilled in the art will recognize that the multiple embodiments disclosed hereinbelow are not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the invention as defined by the appended claims. Any headings used herein are for organizational purposes only and are not meant to limit the scope of the description or the claims. As used herein, the word “can” and “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION

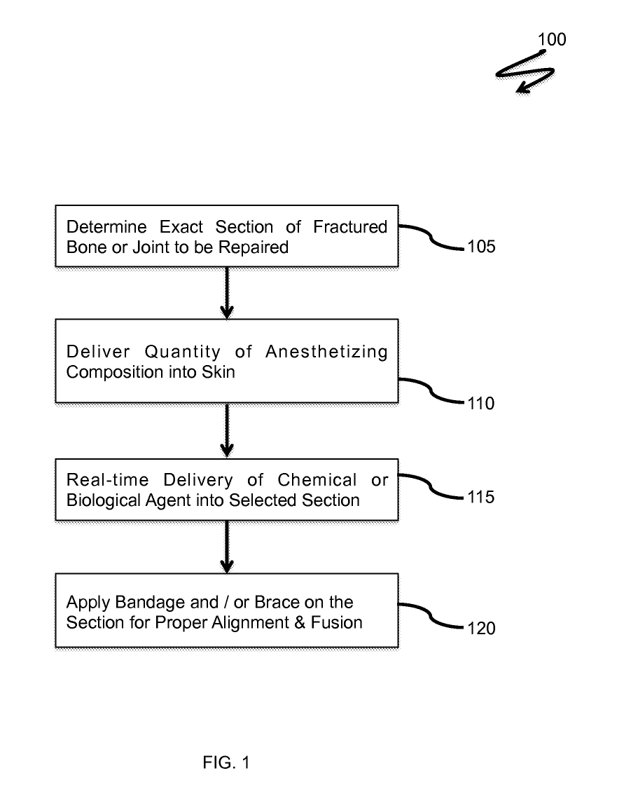

Various embodiments of the present invention relate to methods and kits for repairing one or more fractured bones or fractured/displaced/loose joints of a patient by one or more chemical and/or biological agents. FIG. 1 illustrates a flow diagram of a method 100 for repairing a fractured bone or a joint of a patient by chemical and/or biological agents. In accordance with an embodiment of the present invention, the method 100 includes the steps of, determining an exact section of the fractured bone or the joint to be repaired at step 105. At step 110, a quantity of an anesthetizing composition is selectively delivered into skin surrounding the exact section in order for at least a portion of the anesthetizing composition to be absorbed by one or more tissues in vicinity of one or more nerves responsible for transmitting pain impulses from the exact section to brain of the patient in order to thereby selectively and remotely anesthetize at least a portion of the exact section. This step allows the physician to confirm the problem being treated and simultaneously allows to develop a planned approach to be followed. The method 100 proceeds to step 115. At step 115, the physician supervises the exact section of repair in real-time and one or more chemical or biological agents are delivered into the exact section.

In accordance with an embodiment of the present invention, the step of supervising the exact section in real-time is performed via medical imaging. In use, the medical imaging is selected from a group includes CT scan, ultrasound, X ray, MRI scan, fluoroscopic imaging, and the like depending on patient's presentation, access to imaging facility and product used. The live image guidance ensures safety and precision in delivering the injection to the patient. Moreover, the imaging options can be chosen as noted above as well as chemical and/or biological agents chosen depending on the indication and presentation of the condition.

The method 100 proceeds to step 120. At step 120, one or more bandages and/or one or more braces are applied on the exact section for proper alignment and fusion of the bone or joint. Moreover, the stabilization of the body part is the key to ensure correct fusion and healing.

In accordance with an embodiment of the present invention, the chemical and/or biological agent is delivered into the exact section by inserting a needle. In use, the method further includes the step of controlling an amount of the chemical or biological agent to be delivered into the exact section. Particularly, the correct type and amount of injectate is to be inserted into the joint or bone is assessed by the physician depending on the size and nature of problem.

In accordance with an embodiment of the present invention, the method 100 further includes the step of changing approach of delivering the chemical and/or biological agent into the exact section depending on presence of anatomical variations or access to target site issues. In use, the chemical or biological agent is delivered into the exact section uniformly across surface of the fractured bone or the joint to be repaired.

In accordance with an embodiment of the present invention, the biological agents may include but not limited to, Bone Morphogenic Protein (BMP), growth factors, stem cells, interleukins, cytokines or any other autologous or allogenic product derived from living organism or cadaver or the like. In use, the biological agent may further include a statins. Generally, bone tissues have tendency to regenerate to native tissue in response to injury. However, the fracture repair process may require a viable biological microenvironment to ensure successful healing to native tissue, wherein multiple biologic agents may augment the biological microenvironment and enhance bone repair. The chemical agents may include but not limited to polymers, synthetic materials or scaffolds that would allow individually or in combination with biologic agents to create fusion or adhesion between bones and joints.

In accordance with an embodiment of the present invention, a kit for repairing a fractured bone or a joint of a patient by chemical and/or biological agent includes, an anesthetizing device for selectively delivering a quantity of an anesthetizing composition into skin surrounding an exact section of the fractured bone or the joint to be repaired in order for at least a portion of the anesthetizing composition to be absorbed by one or more tissues in vicinity of one or more nerves responsible for transmitting pain impulses from the exact section to brain of the patient in order to thereby selectively and remotely anesthetize at least a portion of the exact section, at least one chemical or biological agent, and, a delivery device for delivering the chemical or biological agent into the exact section.

In accordance with an embodiment of the present invention, the kit further includes, a medical imaging module for supervising the exact section in real-time, and, one or more bandages and/or one or more braces for applying on the exact section for proper alignment and fusion. In use, the delivery device is an injection device. Those of ordinary skill in the art will appreciate that the medical imaging module is selected from a group includes CT scan, ultrasound, X ray, MRI scan, fluoroscopic imaging, and the like. The imaging study at Step 105 is optional between CT, X ray, and MRI scan.

Therefore, as may be seen, embodiments of the present invention provide method and kit for repairing a fractured bone or a joint of a patient without any surgical manipulation. Moreover, the recovery time of the patient and post-operative pain is reduced significantly. As no metal is utilized in the present method to achieve the fusion, there will be no device or hardware outside the margins of the bone or joint. Accordingly, the fusion will happen within the bone or joint maintaining normal external morphology of the bone or joint. Further, there is no incision of skin and the fusion is going to be accomplished via injection or minimally invasive method. The present method is more physiologic in nature than the one produced by utilizing metal component and by following the mentioned steps the fusion can be safely and effectively accomplished by the physician. The present method and kit can be used for joints or bones anywhere in the body as well as other tissues that need opposition i.e. bringing close together in order to heal.

Accordingly, while there has been shown and described the preferred embodiment of the invention is to be appreciated that the invention may be embodied otherwise than is herein specifically shown and described and, within said embodiment, certain changes may be made in the form and arrangement of the parts without departing from the underlying ideas or principles of this invention within the scope of the claims appended herewith.

What is claimed is:

1. A method for repairing a fractured bone or a joint of a patient by chemical and/or biological agents, said method comprising the steps of:
determining an exact section of said fractured bone or said joint to be repaired;
selectively delivering a quantity of an anesthetizing composition into skin surrounding said exact section in order for at least a portion of said anesthetizing composition to be absorbed by at least one tissue in vicinity of at least one nerve responsible for transmitting pain impulses from said exact section to brain of said patient in order to thereby selectively and remotely anesthetize at least a portion of said exact section;
supervising said exact section in real-time and delivering at least one chemical and/or biological agent into said exact section; and
applying at least one bandage and/or at least one brace on said exact section for proper alignment and fusion.

2. The method as claimed in claim 1, wherein said at least one chemical and/or biological agent is delivered into said exact section by inserting a needle.

3. The method as claimed in claim 1, wherein said method further comprises the step of controlling an amount of said at least one chemical and/or biological agent to be delivered into said exact section.

4. The method as claimed in claim 1, wherein said method further comprises the step of changing approach of delivering said at least one chemical or biological agent into said exact section depending on presence of anatomical variations or access to target site issues.

5. The method as claimed in claim 1, wherein said at least one chemical and/or biological agent is delivered into said exact section uniformly across surface of said fractured bone or said joint to be repaired.

6. The method as claimed in claim 1, wherein said step of supervising said exact section in real-time is performed via medical imaging.

7. The method as claimed in claim 6, wherein said medical imaging is selected from a group comprising CT scan, ultrasound, X ray, MRI scan, fluoroscopic imaging, and the like.

8. The method as claimed in claim 1, wherein said at least one biological agent is selected from a group comprising Bone Morphogenic Protein (BMP), growth factors, stem cells, interleukins, cytokines, any other autologous or allogenic product derived from living organism or cadaver and the like.

9. The method as claimed in claim 1, wherein said at least one biological agent comprises a statins.

10. The method as claimed in claim 1, wherein said at least one chemical agent is selected from a group comprising polymers, synthetic materials or scaffolds that would allow individually or in combination with said at least one biological agent to create fusion or adhesion between bones and joints.

11. A kit for repairing a fractured bone or a joint of a patient by chemical and/or biological agents, said kit comprising:
an anesthetizing device for selectively delivering a quantity of an anesthetizing composition into skin surrounding an exact section of said fractured bone or said joint to be repaired in order for at least a portion of said anesthetizing composition to be absorbed by at least one tissue in vicinity of at least one nerve responsible for transmitting pain impulses from said exact section to brain of said patient in order to thereby selectively and remotely anesthetize at least a portion of said exact section;
at least one chemical or biological agent; and,
a delivery device for delivering said at least one chemical or biological agent into said exact section.

12. The kit as claimed in claim 10, wherein said kit further comprises a medical imaging module for supervising said exact section in real-time.

13. The kit as claimed in claim 10, wherein said kit further comprises at least one bandage and/or at least one brace for applying on said exact section for proper alignment and fusion.

13. The kit as claimed in claim 10, wherein said delivery device is an injection or a minimally invasive device.

14. The kit as claimed in claim 11, wherein said medical imaging module is selected from a group comprising CT scan, ultrasound, X ray, MRI scan, fluoroscopic imaging, and the like.

15. The kit as claimed in claim 10, wherein said at least one biological agent is selected from a group comprising Bone Morphogenic Protein (BMP), growth factors, stem cells, interleukins, cytokines, any other autologous or allogenic product derived from living organism or cadaver and the like.

16. The kit as claimed in claim 10, wherein said at least one biological agent comprises a statins.

17. The kit as claimed in claim 10, wherein said at least one chemical agent is selected from a group comprising polymers, synthetic materials or scaffolds that would allow individually or in combination with said at least one biological agent to create fusion or adhesion between bones and joints.

18. The kit as claimed in claim 10, wherein said kit further comprises a plurality of instructions.

19. The kit as claimed in claim 10, wherein said plurality of instructions comprises:
determining said exact section of said fractured bone or said joint to be repaired;
selectively delivering said quantity of said anesthetizing composition into skin surrounding said exact section in order for at least a portion of said anesthetizing composition to be absorbed by at least one tissue in vicinity of at least one nerve responsible for transmitting pain impulses from said exact section to brain of said patient in order to thereby selectively and remotely anesthetize at least a portion of said exact section;
supervising said exact section in real-time and delivering at least one chemical and/or biological agent into said exact section; and
applying at least one bandage and/or at least one brace on said exact section for proper alignment and fusion.

19. The kit as claimed in claim 18, wherein said plurality of instructions further comprises the step of controlling an amount of said at least one chemical and/or biological agent to be delivered into said exact section.

20. The kit as claimed in claim 18, wherein said plurality of instructions further comprises the step of changing approach of delivering said at least one chemical and/or biological agent into said exact section depending on presence of anatomical variations or access to target site issues.
